(19) United States
(12) Patent Application Publication
(10) Pub. No.: US 2026/0272648 A1
(43) Pub. Date: Sep. 17, 2026

(54) DELIVERY SYSTEMS FOR PROSTHETIC HEART VALVES

(71) Applicant: Edwards Lifesciences Corporation, Irvine, CA (US)

(72) Inventors: Grant Jason Gangeness, Carlsbad, CA (US); Tri D. Tran, Fountain Valley, CA (US); Ronaldo C. Cayabyab, Mission Viejo, CA (US); Yidong M. Zhu, Tustin, CA (US)

(21) Appl. No.: 19/672,385

(22) Filed: May 8, 2026

Related U.S. Application Data
(63) Continuation of application No. PCT/US2024/054662, filed on Nov. 6, 2024.
(60) Provisional application No. 63/598,049, filed on Nov. 10, 2023.

(51) Int. Cl. A61F 2/24

(52) U.S. Cl. CPC A61F 2/2433

(57) ABSTRACT

A delivery apparatus can include a handle, a first shaft extending distally from the handle, a second shaft extending distally from the handle coaxially through the first shaft, a shoulder assembly disposed at a distal end portion of the delivery apparatus, and a balloon coupled to a distal end of the second shaft. The balloon can extend over the shoulder assembly and can be shape set to form a distal balloon bump over the shoulder assembly, a central portion, and a proximal balloon bump disposed proximally of the central portion.

### CROSS REFERENCE TO RELATED APPLICATION

This application is a continuation of PCT Patent Application No. PCT/US2024/054662, filed on November 6, 2024, which claims the benefit of U.S. Provisional Application No. 63/598,049, filed November 10, 2023. The entire disclosure of each of these applications are incorporated herein by reference in its entirety.

### FIELD

The present disclosure concerns examples of delivery systems for prosthetic implants.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

### SUMMARY

Described herein are a delivery apparatus, and methods for implanting prosthetic heart valves. The disclosed delivery apparatus, and methods can, for example, (1) provide uniform inflation for balloon-expandable prosthetic valves, (2) reduce gaps between a prosthetic valve and the delivery apparatus (e.g., to prevent or mitigate snagging of the valve on the native anatomy during implantation), (3) correctly orient a prosthetic valve relative to the delivery apparatus, (4) ensure retraction of an outer shaft to prevent constraint of the balloon during inflation, and/or (5) provide a desired rigidity/flexibility to the delivery apparatus to facilitate tracking through the patient’s anatomy while ensuring protection of the inflation lumen within the delivery apparatus. As such, the devices and methods disclosed herein can, among other things, overcome one or more of the deficiencies of typical prosthetic heart valves and their delivery apparatus.

A delivery apparatus for a prosthetic implant can comprise a handle and one or more shafts coupled to the handle.

In some examples, a delivery apparatus can comprise a shoulder assembly disposed at a distal end portion thereof.

In some examples, a delivery apparatus can comprise a balloon shape set to form a distal balloon bump, a central portion, and a proximal balloon bump.

In some examples, a radially expandable and compressible prosthetic valve can be disposed at least partially over a central portion of the balloon in a radially compressed configuration such that a first end portion of the prosthetic valve is crimped partially over a distal end portion of the proximal balloon bump.

In some examples, the prosthetic valve can include a positioning member disposed on an outflow end of the prosthetic valve, wherein when the prosthetic heart valve is disposed on the balloon the positioning member is adjacent the shoulder assembly.

In some examples, a delivery apparatus can comprise a second shaft comprising a plurality of longitudinally extending sections including a first section, a second section, a third section, a fourth section, and a fifth section.

In some examples, the first section is more rigid than the second, third, fourth, and fifth sections.

In some examples, the first, fourth, and fifth sections are more rigid than the second and third sections.

In some examples, a first shaft of the delivery apparatus comprises a first marker band disposed at a distal end thereof, and a second shaft of the delivery apparatus comprises a second marker band disposed at a distal end portion thereof adjacent a proximal end of the balloon.

In some examples, a delivery apparatus comprises a handle; a first shaft extending distally from the handle; a second shaft extending distally from the handle coaxially through the first shaft; a shoulder assembly disposed at a distal end portion of the delivery apparatus; and a balloon coupled to a distal end of the second shaft and extending over the shoulder assembly, the balloon being shape set to form a distal balloon bump over the shoulder assembly, a central portion, and a proximal balloon bump disposed proximally of the central portion.

In some examples, a delivery apparatus comprises a handle; a first shaft extending distally from the handle; a second shaft extending distally from the handle coaxially through the first shaft; a third shaft extending distally from the handle coaxially through the first and second shafts; a shoulder assembly coupled to a distal end portion of the third shaft; and a balloon coupled to a distal end of the second shaft and extending over the shoulder assembly, the balloon being shape set to form a distal balloon bump over the shoulder assembly, a central portion, and a proximal balloon bump disposed proximally of the central portion.

A delivery system for implanting one or more prosthetic implants can generally comprise a guide catheter, and one or more prosthetic implant delivery apparatuses. In addition to these components, a delivery system can further comprise one or more of the components disclosed herein.

A guide catheter can comprise a handle and one or more shafts coupled to the handle.

A prosthetic implant delivery apparatus can comprise a handle and one or more shafts coupled to the handle.

In some examples, a delivery assembly can comprise a guide catheter comprising a handle and a guide shaft extending distally from the handle; and a delivery apparatus configured to be advanced through the guide catheter, the delivery apparatus comprising: a handle;

a first shaft extending distally from the handle; a second shaft extending distally from the handle coaxially through the first shaft; a third shaft extending distally from the handle coaxially through the first and second shafts; a shoulder assembly coupled to a distal end portion of the third shaft; and a balloon coupled to a distal end of the second shaft and extending over the shoulder assembly, the balloon being shape set to form a distal balloon bump over the shoulder assembly, a central portion, and a proximal balloon bump disposed proximally of the central portion.

In some examples, a delivery apparatus or delivery assembly including a delivery apparatus comprises one or more of the components recited in Examples 1-109 below.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a stage in an example mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the example mitral valve replacement procedure where a docking device delivery apparatus extending through the guide catheter is implanting a docking device for a prosthetic heart valve at the native mitral valve.

FIG. 2B schematically illustrates another stage in the example mitral valve replacement procedure where the docking device of FIG. 2A is fully implanted at the native mitral valve of the patient and the docking device delivery apparatus has been removed from the patient.

FIG. 3A schematically illustrates another stage in the example mitral valve replacement procedure where a prosthetic heart valve delivery apparatus extending through the guide catheter is implanting a prosthetic heart valve in the implanted docking device at the native mitral valve.

FIG. 3B schematically illustrates another stage in the example mitral valve replacement procedure where the prosthetic heart valve is fully implanted within the docking device at the native mitral valve and the prosthetic heart valve delivery apparatus has been removed from the patient.

FIG. 4 schematically illustrates another stage in the example mitral valve replacement procedure where the guide catheter and the guidewire have been removed from the patient.

FIG. 5 is side view of an exemplary guide catheter configured to receive a delivery apparatus and guide the delivery apparatus through a portion of a patient’s vasculature, according to an example.

FIG. 6 is a perspective view of a delivery apparatus for a prosthetic heart valve, according to an example.

FIG. 7 is a perspective view of a prosthetic heart valve for use with the delivery apparatus of FIG. 6, according to an example.

FIG. 8 is a perspective view of a distal end portion of a delivery apparatus for a prosthetic heart valve, according to an example.

FIG. 9 is an enlarged view of a portion of the delivery apparatus of FIG. 8.

FIG. 10 is a perspective view of the delivery apparatus of FIG. 8 with a prosthetic heart valve mounted thereon in a radially compressed configuration.

FIG. 11 is a perspective view of the delivery apparatus of FIG. 10 including the frame of the prosthetic heart valve of FIG. 10.

FIG. 12 is a side view of an inflatable balloon for a delivery apparatus, shown in the inflated configuration.

FIG. 13 is a cross sectional side view of the inflatable balloon of FIG. 12.

FIG. 14 is a side view of a catheter for use with a delivery apparatus, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient’s body), while distal motion of the device is motion of the device away from the user and toward the implantation site (e.g., into the patient’s body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

Described herein are examples of a steerable delivery apparatus (sometimes referred to as a steerable catheter) that can be used to navigate a subject’s vasculature to deliver an implantable, expandable medical device (e.g., a prosthetic heart valve), tools, agents, or other therapy to a location within the body of a subject. Examples of procedures in which the steerable catheters are useful include neurological, urological, gynecological, fertility (e.g., in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

In connection therewith, various systems, apparatuses, methods, or the like are described herein that, in some examples, can help ensure the expandable medical device is expanded to its functional size when implanted within the body of the subject.

A defective native heart valve may be replaced with a transcatheter prosthetic heart valve in a transcatheter heart valve replacement procedure. Typically, the prosthetic heart valve can be implanted within a native valve annulus of the defective native heart valve using a delivery apparatus (which is also referred to herein as a “prosthetic heart valve delivery apparatus” and/or a “balloon catheter”). Typically, the delivery apparatus can comprise a shaft and an inflatable balloon mounted to a distal end portion of the shaft. The prosthetic heart valve can be mounted around the balloon, which can inflate to radially expand the prosthetic heart valve at the native valve annulus.

However, in some examples, the prosthetic heart valve may not be able to sufficiently conform to the geometry of the native tissue (for example, to the leaflets and/or annulus of the native heart valve) and may undesirably shift around relative to the native tissue, which can lead to paravalvular leakage. Thus, a docking device may be implanted first at the native valve annulus and then the prosthetic heart valve can be implanted within the docking device to help anchor the prosthetic heart valve to the native tissue and provide a seal between the native tissue and the prosthetic heart valve.

### Examples of the Disclosed Technology

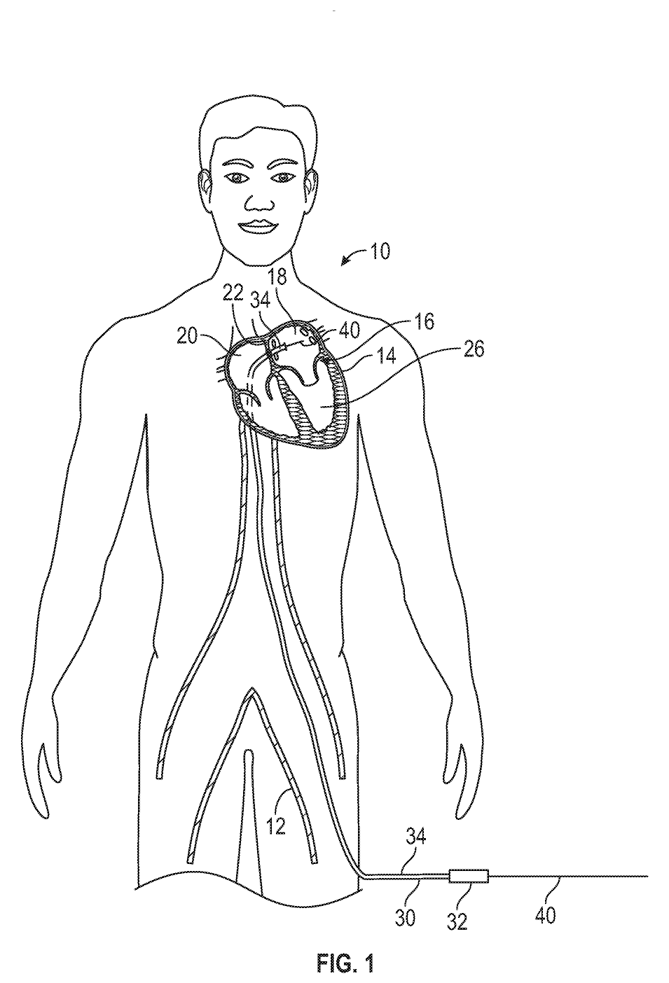

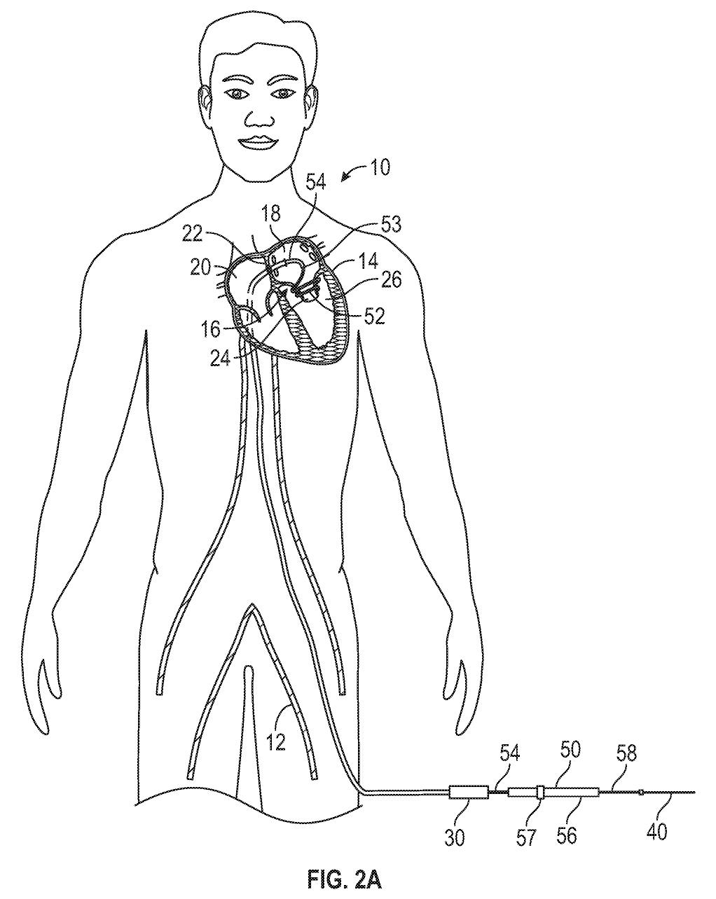

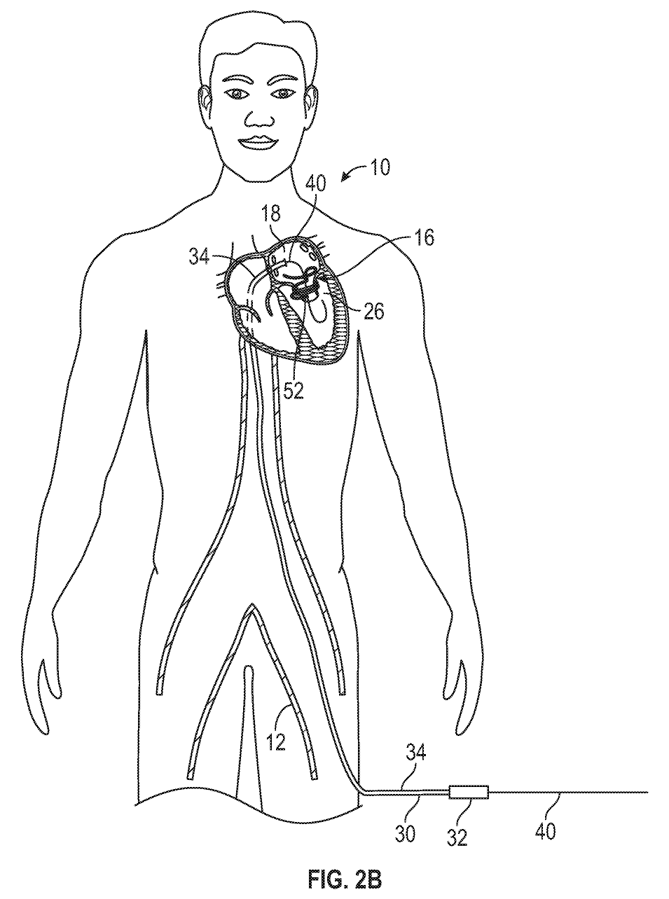

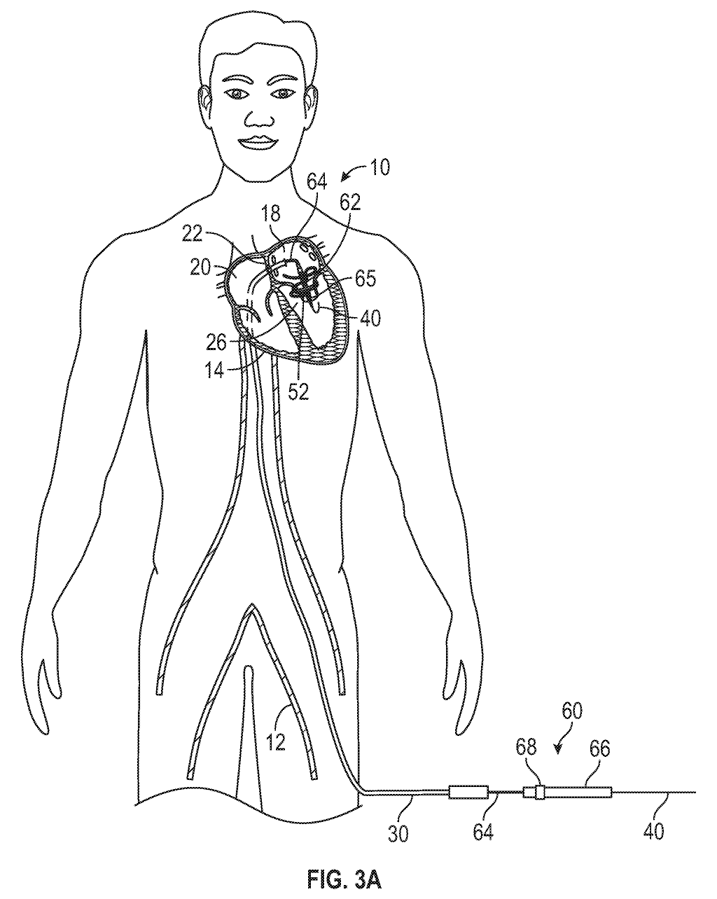

FIGS. 1-4 depict an example of a transcatheter heart valve replacement procedure (for example, a mitral valve replacement procedure) which utilizes a docking device 52 and a prosthetic heart valve 62, according to one example. During the procedure, a user first creates a pathway to a patient’s native heart valve using a guide catheter 30 (FIG. 1). The user then delivers and implants the docking device 52 at the patient’s native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (for example, the native mitral valve 16 or native mitral valve annulus). As shown, the heart 14 is illustrated schematically. For example, the anterior leaflet and chordae of the native mitral valve 16 are omitted for illustration purposes, such that only a portion of the posterior leaflet of the native mitral valve 16 is illustrated.

Initially, the user may first make an incision in the patient’s body to access the blood vessel 12. For example, in the example illustrated in FIG. 1, the user may make an incision in the patient’s groin to access a femoral vein. Thus, in such examples, the blood vessel 12 may be a femoral vein.

After making the incision at the blood vessel 12, the user may insert the guide catheter 30, the guidewire 40, and/or additional devices (for example, an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (for example, the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (for example, the guide catheter 30, the docking device delivery apparatus 50, the prosthetic valve delivery apparatus 60, additional catheters, or the like) and their associated devices (for example, docking device, prosthetic heart valve, and the like) to the implantation site within the heart 14, and thus may extend all the way through the blood vessel 12 and into a left atrium 18 of the heart 14 (FIG. 1) and in some examples, through the native mitral valve 16 and into a left ventricle of the heart 14.

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (for example, through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient’s vasculature (blood vessel 12) and to the implantation site (for example, the native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient’s vasculature (for example, the blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (for example, the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO 2020/247907, which is incorporated by reference herein in its entirety.

Referring again to FIG. 2A, after the guide catheter 30 is positioned within the left atrium 18, the user may insert the docking device delivery apparatus 50 (for example, the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (for example, pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (for example, the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (for example, the portion of the docking device 52 shown in FIG. 2A that is configured to be positioned within a left ventricle 26 and/or on the ventricular side of the native mitral valve 16), the user may then deploy the remaining portion of the docking device 52 (for example, an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient’s vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient’s vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (for example, the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, inflate the inflatable balloon), thereby radially expanding the prosthetic heart valve 62 within the docking device 52.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (for example, tricuspid, pulmonary, and/or aortic valves). Further, the same and/or similar delivery apparatuses (for example, docking device delivery apparatus 50, prosthetic valve delivery apparatus 60, guide catheter 30, and/or guidewire 40), docking devices (for example, docking device 52), replacement heart valves (for example, prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient’s vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

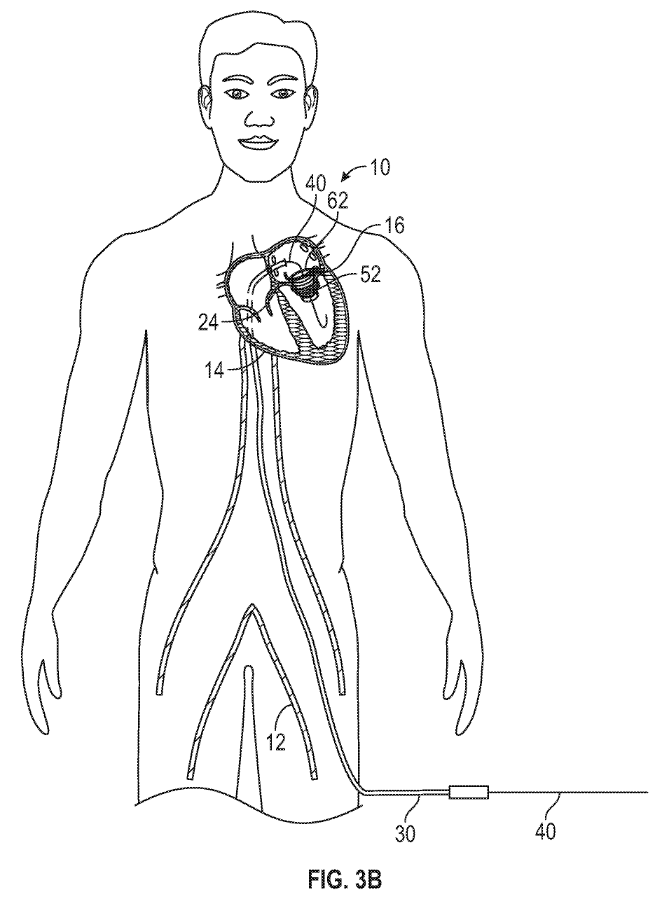

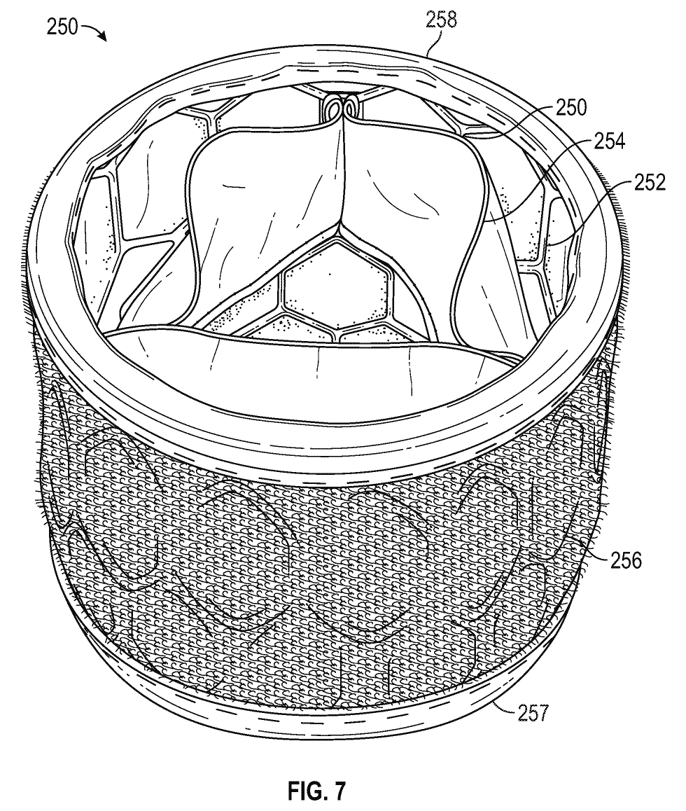

FIG. 5 illustrates an exemplary guide catheter, which is referred to below as a guide sheath 100 (and can also be referred to herein as a “delivery apparatus” or an “introducer device”). The guide sheath 100 can be configured to be inserted into a patient’s vasculature and receive a balloon catheter or delivery apparatus therein in order to introduce the balloon catheter into the patient’s vasculature and at least partially guide the balloon catheter therein to a target implantation site. For example, the guide sheath 100 can be used as the guide sheath 30 in a prosthetic valve implantation procedure, as described above with reference to FIGS. 1-4. An exemplary balloon catheter for a prosthetic medical device (referred to below as “delivery apparatus 400”) that can be received within the guide sheath 100 is shown in FIG. 9, as described further below. Though the guide sheath 100 is described herein as being used with the delivery apparatus 400, the guide sheath 100 can be configured to receive a variety of delivery apparatuses, balloon catheters, or implant catheters, such as alternate prosthetic heart valve delivery apparatuses, docking device delivery apparatuses, and/or delivery apparatuses for other prosthetic medical devices or medical therapies, such as stents.

The guide sheath 100 in the illustrated example comprises a handle 102, an elongated shaft 104 extending distally from the handle 102, and a central longitudinal axis 112. The shaft 104 has a main (or primary) lumen that is defined by an inner surface of a wall of the shaft 104. The main lumen is configured to receive a delivery apparatus therein (such as any of the prosthetic device delivery apparatuses or implant catheters described herein). In some examples, the shaft 104 can extend into the handle 102. Further, in some examples, the main lumen can extend through the handle 102 to an inlet port 106 disposed at a proximal end of the handle 102. Thus, in some examples, an inner surface of a wall of a portion of the handle (for example, at the proximal end) can further define the main lumen. Thus, the main lumen can extend from the inlet port 106 to a distal end 108 of the shaft 104.

The handle 102 can have an outer housing 105 and can further include a seal housing assembly 110 (which can also be referred to as a “seal stack”) which comprises one or more seals contained therein. The one or more seals of the seal housing assembly 110 can be configured to fluidly seal the main lumen of the guide sheath 100 from the external environment. For example, the one or more seals of the seal housing assembly 110 can be configured to prevent blood from a patient in which the guide sheath 100 is inserted from exiting the guide sheath 100 and prevent air from the environment from entering the guide sheath 100 (for example, through the inlet port 106). The one or more seals can include a variety of types of seals, such as a duckbill seal, a flapper seal, an umbrella valve, a cross-slit valve, a dome valve, or the like.

The handle 102 can, in some instances, include an adaptor spine 114 disposed adjacent and distal to the seal housing assembly 110. A flush port 116 can be connected to the outer housing 105 at the adaptor spine 114. A flush lumen of the adaptor spine 114 is connected to the flush port 116 and further connects to the main lumen. The flush port 116 can be configured to receive fluid through a lumen thereof. In this way, the flush port 116 can be fluidly coupled to the main lumen by the flush lumen.

The handle 102 can include a steering mechanism configured to adjust the curvature of the distal end portion of the shaft 104 (as such, the shaft 104 can be referred to as a steerable shaft). In the illustrated example, the handle 102 includes a main body portion 118 disposed adjacent and distal to the adaptor spine 114 and an adjustment member, such as the illustrated rotatable knob 120. The main body portion 118 can house internal flex mechanisms of the guide sheath 100 which are operatively coupled to the rotatable knob 120. In some examples, the flex mechanisms, and thus the knob 120, can be operatively coupled to the proximal end portion of a pull wire. The pull wire can extend distally from the handle 102 through the shaft 104 and have a distal end portion affixed to the shaft 104 at or near the distal end 108 of the shaft 104. Rotating the knob 120 can increase or decrease the tension in the pull wire, thereby adjusting the curvature of the distal end portion of the shaft 104. Further details on steering or flex mechanisms for a delivery apparatus can be found in U.S. Patent No. 9,339,384, which is incorporated by reference herein.

A delivery apparatus configured to implant a docking device (referred to herein as a “dock delivery catheter” or “dock delivery system”), can be used in conjunction with the guide sheath 100. For example, the dock delivery catheter can be used at the docking device delivery apparatus 50 in a prosthetic valve implantation procedure, as described above with reference to FIG. 2A. Further details on delivery apparatus/catheters/systems (including various examples of the handle assembly) that are configured to deliver a docking device to a target implantation site can be found in International Publication No. WO 2020/247907 and in U.S. Patent Publication Nos. 2018/0318079 and 2018/0263764, all of which are incorporated by reference herein in their entireties.

Further details of the docking device (such as docking device 52) and its variants are described in International Publication No. WO 2022/087336, which is incorporated by reference herein in its entirety, and International Publication No. WO 2020/247907.

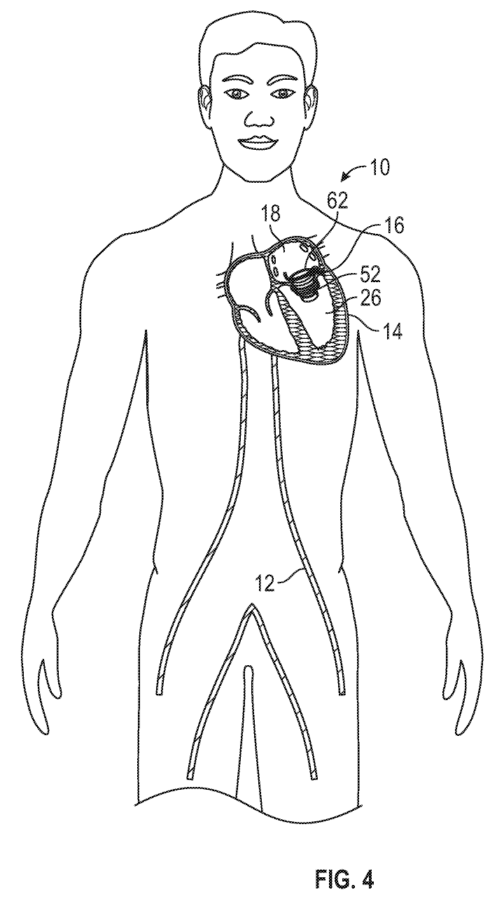

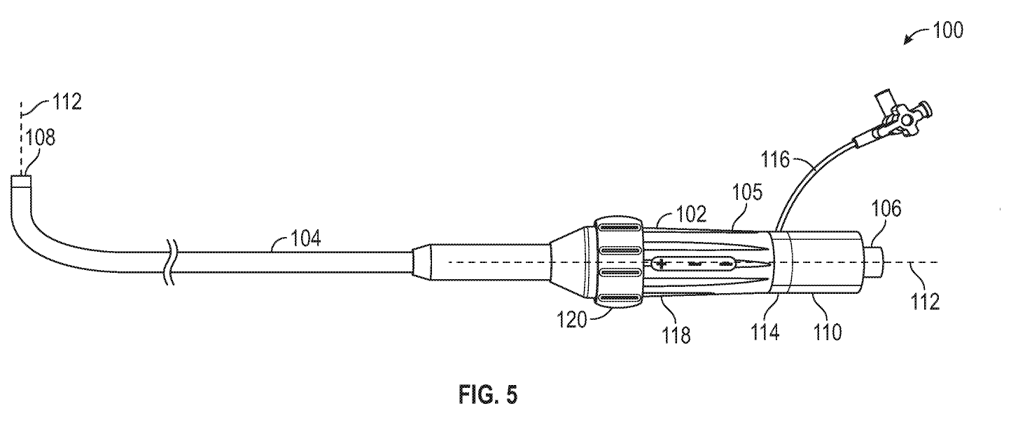

FIG. 6 illustrates a prosthetic heart valve delivery apparatus 200 (which can also be referred to here as an “implant catheter” and/or a “balloon catheter”) that can be used to implant an expandable prosthetic heart valve 250 (FIG. 7), according to one example. In some examples, the delivery apparatus 200 is specifically adapted for use in introducing a prosthetic heart valve into a heart. For example, the delivery apparatus 200 can be used as the prosthetic heart valve delivery apparatus 60 in a prosthetic valve implantation procedure, as described above with reference to FIG. 3A.

The delivery apparatus 200 in the illustrated example of FIG. 6 is a balloon catheter comprising a handle 202 and a steerable, outer shaft 204 extending distally from the handle 202. The delivery apparatus 200 can further comprise an intermediate shaft 206 (which also may be referred to as a “balloon shaft”) that extends proximally from the handle 202 and distally from the handle 202, the portion extending distally from the handle 202 also extending coaxially through the outer shaft 204. In some examples, the delivery apparatus 200 can further comprise an inner shaft extending distally from the handle 202 coaxially through the intermediate shaft 206 and the outer shaft 204 and proximally from the handle 202 coaxially through the intermediate shaft.

The outer shaft 204 and the intermediate shaft 206 can be configured to translate (e.g., move) longitudinally, along a central longitudinal axis 220 of the delivery apparatus 200, relative to one another to facilitate delivery and positioning of a prosthetic valve at an implantation site in a patient’s body.

The intermediate shaft 206 can include a proximal end portion that extends proximally from a proximal end of the handle 202, to an adaptor 212. The adaptor 212 can include a first port 238 configured to receive a guidewire therethrough and a second port 240 configured to receive fluid (e.g., inflation fluid) from a fluid source. The second port 240 can be fluidly coupled to an inner lumen of the intermediate shaft 206.

In some examples, the intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft can extend distally beyond the distal end portion of the intermediate shaft 206 toward or to a nose cone 222 at a distal end of the delivery apparatus 200.

In some examples, a distal end of the balloon 218 can be coupled to a distal end of the delivery apparatus 200, such as to the nose cone 222 (as shown in FIG. 6), or to an alternate component at the distal end of the delivery apparatus 200 (for example, a distal shoulder). An intermediate portion of the balloon 218 can overlay a valve mounting portion 224 of a distal end portion of the delivery apparatus 200 and a distal end portion of the balloon 218 can overly a distal shoulder of the delivery apparatus 200. As shown in FIG. 6, a prosthetic heart valve 250 can be mounted around the balloon 218, at the valve mounting portion 224 of the delivery apparatus 200, in a radially compressed state. The prosthetic heart valve 250 can be configured to be radially expanded by inflation of the balloon 218 at a native valve annulus, as described above with reference to FIG. 3A.

A balloon shoulder assembly of the delivery apparatus 200, which includes the distal shoulder, is configured to maintain the prosthetic heart valve 250 (or other medical device) at a fixed position on the balloon 218 during delivery through the patient’s vasculature.

The outer shaft 204 can include a distal tip portion 226 mounted on its distal end. In some examples, the outer shaft 204 and the intermediate shaft 206 can be translated axially relative to one another to position the distal tip portion 226 adjacent a proximal end of the valve mounting portion 224, when the prosthetic valve 250 is mounted in the radially compressed state on the valve mounting portion 224 (as shown in FIG. 6) and during delivery of the prosthetic valve to the target implantation site. As such, the distal tip portion 226 can be configured to resist movement of the prosthetic valve 250 relative to the balloon 218 proximally, in the axial direction, relative to the balloon 218, when the distal tip portion 226 is arranged adjacent a proximal side of the valve mounting portion 224.

An annular space can be defined between an outer surface of the inner shaft and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft and an inner surface of the balloon 218. As such, fluid from the fluid source can flow to the fluid passageway from the annular space to inflate the balloon 218 and radially expand and deploy the prosthetic valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Patent No. 9,339,384. In some examples, the handle 202 can comprise no integrated steering mechanism, but rather can be steered using the guide sheath 100.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (for example, for fine positioning at the implantation site).

Prosthetic valves disclosed herein (for example, prosthetic heart valve 450, prosthetic heart valve 62, etc.) can be radially compressible and expandable between a radially compressed state and a radially expanded state. Thus, the prosthetic valves can be crimped on or retained by an implant delivery apparatus (for example, delivery apparatus 200, delivery apparatus 60, etc.) in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

FIG. 7 illustrates the prosthetic valve 250 in a radially expanded position. The prosthetic valve 250 can be used as the prosthetic heart valve 62 in a prosthetic valve implantation procedure, as described above with reference to FIGS. 1-4. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device (for example, docking device 52, etc.) that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in International Publication No. WO2020/247907. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 250 can be used as the prosthetic heart valve 62 in a prosthetic valve implantation procedure, as described above with reference to FIGS. 1-4. As shown in FIG. 7, the prosthetic valve 250 can include a frame 252 and a plurality of leaflets 254 situated at least partially within the frame 252. The prosthetic valve 250 can also include an outer covering 256 (which is also referred to herein as an “outer skirt”) situated about the frame 252. As shown in FIG. 7, the prosthetic valve 250 includes an inflow end 257 and an outflow end 258. The terms “inflow” and “outflow” are related to the normal direction of blood flow (for example, antegrade blood flow) through the prosthetic valve 250. For example, the leaflets 254 can allow blood flow through the valve 250 in a direction from the inflow end 257 to the outflow end 258 and prevent the reverse flow (for example, prevent flow in a direction from the outflow end 258 to the inflow end 257).

The frame 252 can be made of any of various suitable plastically-expandable materials (for example, stainless steel, etc.) or self-expanding materials (for example, Nitinol) as known in the art. When constructed of a plastically-expandable material, the frame 252 (and thus the valve 250) can be crimped to a radially compressed state on a delivery catheter and then expanded inside a patient by an inflatable balloon or equivalent expansion mechanism. When constructed of a self-expandable material, the frame 252 (and thus the valve 250) can be crimped to a radially compressed state and restrained in the compressed state by insertion into a sheath or equivalent mechanism of a delivery catheter. Once inside the body, the valve 250 can be advanced from the delivery sheath, which allows the valve 250 to expand to its functional size.

Suitable plastically-expandable materials that can be used to form the frames disclosed herein (for example, the frame 252) include, metal alloys, polymers, or combinations thereof. Example metal alloys can comprise one or more of the following: nickel, cobalt, chromium, molybdenum, titanium, or other biocompatible metal. In some examples, the frame 252 can comprise stainless steel. In some examples, the frame 252 can comprise cobalt-chromium. In some examples, the frame 252 can comprise nickel-cobalt-chromium. In some examples, the frame 252 comprises a nickel-cobalt-chromium-molybdenum alloy, such as MP35N™ (tradename of SPS Technologies), which is equivalent to UNS R30035 (covered by ASTM F562-2). MP35N™/UNS R30035 comprises 35% nickel, 35% cobalt, 20% chromium, and 10% molybdenum, by weight.

The outer skirt 256 can be wholly or partly formed of any suitable biological material, synthetic material (for example, any of various polymers), or combinations thereof. In some examples, the outer skirt 256 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the outer skirt 456 can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the outer skirt 256 can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the outer skirt 256 can comprise a sponge material or foam, such as polyurethane foam. In some examples, the outer skirt 256 can comprise natural tissue, such as pericardium (for example, bovine pericardium, porcine pericardium, equine pericardium, or pericardium from other sources).

Further details of the prosthetic heart valve and its variants are described in U.S. Patent No. 11,185,406, which is incorporated by reference herein in its entirety.

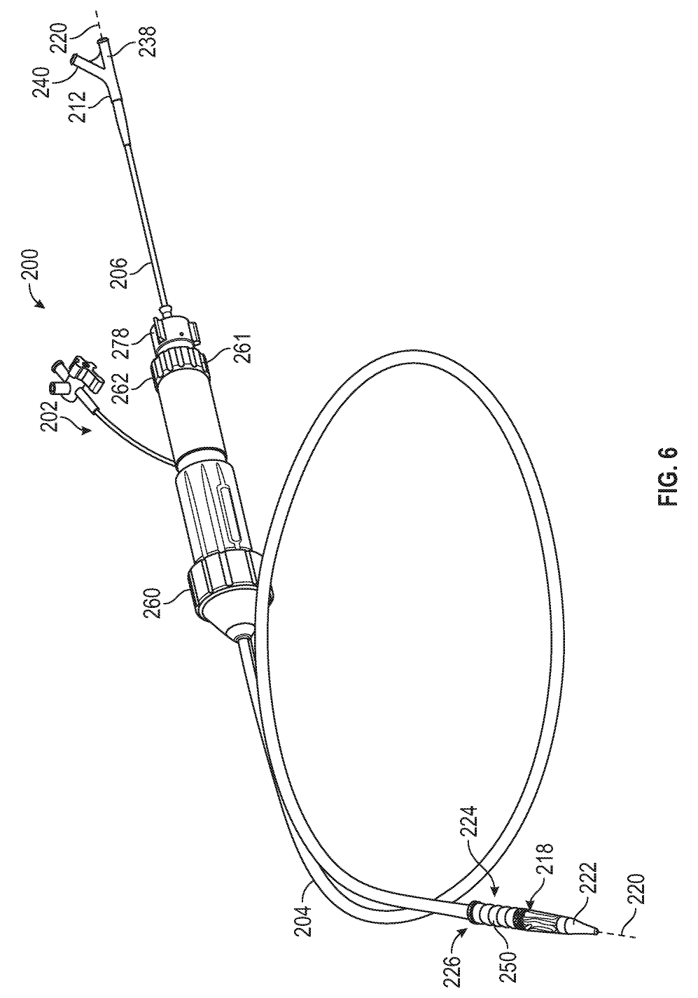

Referring to FIGS. 8-14, in some examples, the delivery apparatus 200 (also referred to as an “implant catheter”) described previously can include a distal end portion 300 in lieu of or in addition to distal tip portion 226. In some examples, the distal end portion 300 can include an inflatable member or balloon 302 in lieu of or in addition to inflatable balloon 218 described previously. The other features of delivery apparatus 200 (such as the handle 202) can be the same as described previously, unless indicated otherwise.

As shown in FIG. 8, the distal end portion 300 of the delivery apparatus 200 can comprise an intermediate shaft 306 (also referred to as a balloon shaft) and an inner shaft 304 extending coaxially through the intermediate shaft 306. The intermediate shaft 306 can include a distal end portion that extends distally beyond a distal end of the outer shaft 204 when a distal end of the outer shaft 204 is positioned away from the inflatable balloon 302. A distal end portion of the inner shaft 304 can extend distally beyond the distal end portion of the intermediate shaft 306 toward or to the nose cone 308 at a distal end of the delivery apparatus. A distal shoulder 310 can be coupled to the distal end portion of the inner shaft 304.

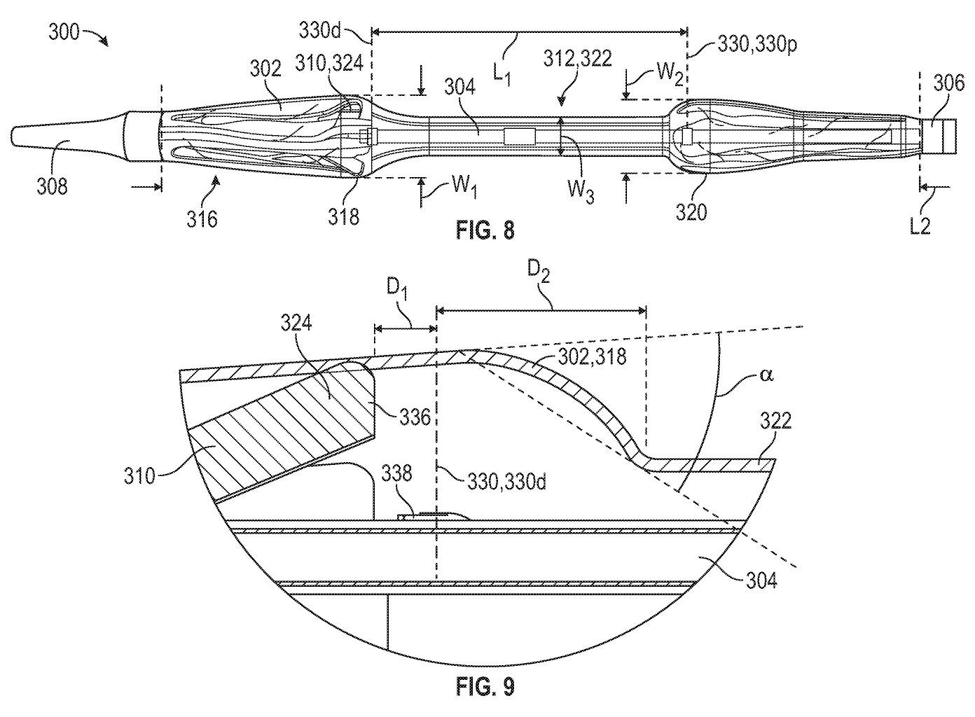

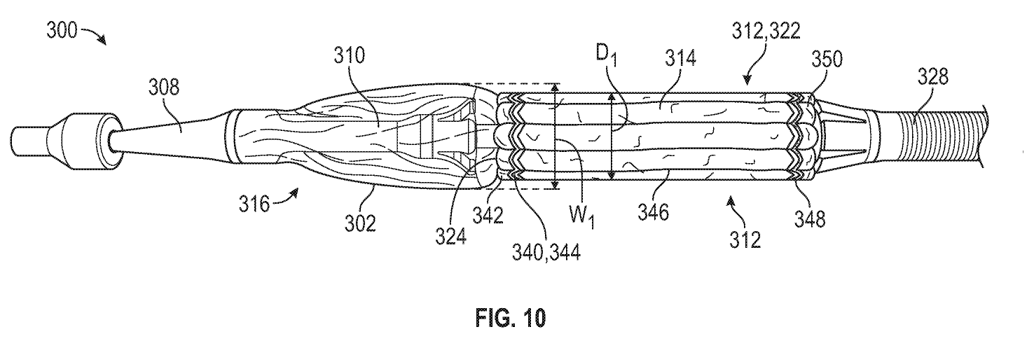

A distal end of the balloon 302 can be coupled to the distal end portion of the delivery apparatus, such as to the nose cone 308, or to an alternate component at the distal end of the delivery apparatus (e.g., distal shoulder 310). As shown in FIG. 8, an intermediate portion of the balloon 302 can overlay a valve mounting portion 312 of the delivery apparatus and a distal end portion of the balloon 302 can be disposed over the distal shoulder 310. A prosthetic heart valve 314, which can be the same or similar to prosthetic valve 250 described previously, can be mounted around the balloon 302 at the valve mounting portion 312, in a radially compressed state, as shown in FIGS. 10-11. The prosthetic heart valve 314 can be configured to be radially expanded by inflation of the balloon 302 at a native valve annulus, as described above with reference to FIGS. 3A and 3B. The distal shoulder 310 can be part of a balloon shoulder assembly 316 of the distal end 300 of the delivery apparatus, which is configured to maintain the prosthetic heart valve 314 at a fixed position on the balloon 302 during delivery through the patient’s vasculature.

Referring to FIG. 8, the balloon shoulder assembly 316 can include the balloon 302, which is formed over a distal end portion of the inner shaft 304 and the distal shoulder 310 to form a distal balloon bump 318 and a proximal balloon bump 320, which define a valve mounting portion 322 (also referred to as a “central portion”) of the balloon 302 between them. The valve mounting portion 322 of the balloon 302 can overlay the valve mounting portion 312 of the delivery apparatus. The balloon 302 can be shape set into this configuration. In some examples, a balloon cover assembly can be disposed over the balloon 302 to maintain the shape set configuration of the balloon prior to use. Further details of the balloon and balloon cover assembly can be found, at least, in International Publication No. WO 2024/015433, which is incorporated by reference herein in its entirety.

The distal balloon bump 318 can have a first width W₁ defined by the width of the flared end portion 324 of the distal shoulder 310, the proximal balloon bump 320 can have a second width W₂, and the valve mounting portion 322 can have a third width W₃ that is less than W₁ and W₂. In some examples, W₂ can be less than W₁, or vice versa. However, in other examples, W₂and W₁ can be substantially equal.

In some examples, the width W₁ of the distal balloon bump can be between about 1 mm and about 10 mm. For example, W₁ can be about 8.75 mm. As shown in FIG. 10, W₁ is configured to be greater than the outer diameter D₁ of the prosthetic valve 314 when the prosthetic valve 314 is crimped on the valve mounting portion 312, 322. This configuration advantageously provides protection for the leading edge 326 of the compressed prosthetic valve 314 as the prosthetic valve 314 exits the guide sheath (such as guide sheath 204) during the implantation procedure and proceeds to the implantation location, such as within a docking device 52. For example, in some examples, the compressed outer diameter D₁ of the prosthetic valve 314 can be about 8.40 mm. The disclosed configurations can also advantageously mitigate radial or axial ‘jumps’ (i.e., unplanned movements of the prosthetic valve) caused by, for example, inherent frame expansion upon retraction of the guide sheath. This can advantageously maximize the physician’s control over the positioning of the prosthetic valve.

In some examples, the width W₃ of the valve mounting portion 322 can be between about 1 mm and about 5 mm. For example, W₃ can be about 4.3 mm. The valve mounting portion 322 can have a length L₁ that aligns with the height of the crimped prosthetic valve 314. For example, the length L₁can be about 31 mm. The valve mounting portion 322 is configured such that during the preparation of the delivery apparatus including distal end portion 300 and accompanying prosthetic valve 314 there is minimal fluid within the valve mounting portion 322, such that the outer diameter of the compressed prosthetic valve 314 is not increased.

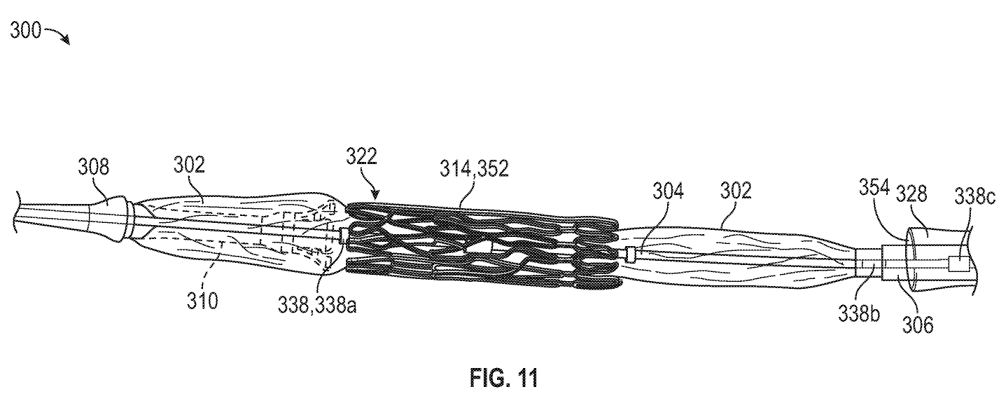

In some examples, the width W₂ of the proximal balloon bump 320 can be between about 1 mm and about 10 mm. For example, W₂ can be about 7.0 mm. The proximal balloon bump 320 can be configured to open simultaneously with the distal cone section 332 (FIG. 12) of the balloon 302, or slightly earlier, this advantageously achieves a uniform “dog bone” inflation effect. The proximal balloon bump 320 advantageously ensures that the prosthetic valve does not embolize by allowing for space/volume upon initial balloon inflation.

In some examples, the prosthetic valve 314 can be crimped slightly over a distal end of the proximal balloon bump 320. This configuration advantageously reduces gaps between the prosthetic valve 314 and the proximal balloon bump 320, which can create a smooth transition for the outer shaft 328 (also called the “flex shaft” in some examples) over the apices of the prosthetic vale and thus prevent or mitigate the prosthetic valve from snagging on the native anatomy during the implantation process. The lack of a proximal shoulder allows the outer shaft 328 (FIG. 10) to easily transition onto and off the compressed prosthetic valve.

In some examples, the total length L₂ (FIG. 8) of the balloon 302 is configured to be as short as possible to facilitate the implantation procedure within the native mitral valve. For example, the length L₂ can be between about 50.0 mm and about 100.0 mm, for example, about 73.0 mm. The total length L₂ can be configured to fit within a balloon cover, such as the balloon cover assembly described in International Publication No. WO2024/015433, which is incorporated by reference herein. This ensures that the balloon is protected during shipping/packaging, and that the shape-set configuration of the balloon is maintained prior to use.

The balloon 302 can comprise one or more witness lines 330 (e.g., a distal witness line 330d and a proximal witness line 330p) that indicate the proximal and distal ends of the central portion 322, see e.g., FIG. 8. In some examples, the witness lines 330d, 330p can be formed by the intersection of the distal and/or proximal cones 332, 334, respectively, with a central portion 322 of the balloon 302, as shown in FIG. 12. The witness lines 330 can be used to align the prosthetic valve 314 on the balloon 302 during the crimping process to ensure that the prosthetic valve 314 is centered on the central portion 322 of the balloon. As shown in FIG. 9, the distal witness line 330d can be spaced a selected distance D₁ from a proximal edge 336 of the distal shoulder 310 to ensure that the central portion 322 of the balloon 302 is centered relative to the crimped prosthetic valve 314. The distance D₁ can be optimized such that when the prosthetic valve 314 is crimped onto the balloon 302 (e.g., using the crimping process taught in International Publication No. WO2024015433), the prosthetic valve 314 is positioned a selected distance from the proximal edge 336 of the distal shoulder 310. This ensures a consistent and safe crimping of the prosthetic valve 314 onto the selected section of the balloon 302. In some examples, the distance D₁ can be between about 1 mm and about 3 mm, for example, the distance D₁ can be about 1.25 mm.

In some examples, the distance D₂ from the distal witness line 330d to a compressed edge of the balloon 302 (e.g., the distal end of the valve mounting portion 322) is configured to create a desirable angle α to ensure that the distal balloon bump 318 has a desired profile. A shorter distance D₂would create a greater angle α, which would undesirably result in a larger distal bump profile. Such a larger distal bump can cause problems during the implantation procedure, for example, due to push forces within the guide sheath 204. In some examples, the angle α can be between about 30 degrees to about 60 degrees. In some examples, the distance D₂ can be between about 3.5 mm and about 5.5 mm, for example, the distance D₂can be about 4.3 mm.

Referring still to FIG. 9, in some examples, the inner shaft 304 can comprise a marker band 338 (e.g., a radiopaque marker band). The prosthetic valve 314 can be crimped onto the balloon 302 such that it lands in a consistent location relative to the marker band 338.

Referring FIG. 10, in some examples, the prosthetic valve 314 can comprise a positioning or orientation member 340 configured to indicate a desired location for an end of the prosthetic heart valve 314 when mounted (and at least partially radially compressed or fully radially compressed) around the valve mounting portion 322 of the balloon 302. For example, the positioning member 340 can be disposed on the outflow end 342 of the prosthetic valve 314 to ensure that the prosthetic valve 314 is crimped on the distal end portion 300 of the delivery apparatus with the outflow end 342 adjacent the distal shoulder 310.

The positioning member 340 can comprise a distinguishing indicium, such as a distinguishing color, shape, or texture. For example, the positioning member 340 can be a colored suture disposed at the outflow end 342 of the prosthetic valve. In some examples, the positioning member 340 can be the suture 344 that is used to couple the outer covering/skirt 346 to the prosthetic valve 314 at the outflow end 342. For example, as shown in FIG. 10, the suture 344 used to couple the outer covering 346 at the outflow end 342 can be an uncolored/white suture, and the suture 348 used to couple the outer covering 346 at the inflow end 350 can be a colored (e.g., green) suture (shown in FIG. 10 using a pattern to indicate a color). In this way the user mounting the prosthetic valve 314 onto the delivery apparatus can visually verify that the prosthetic valve 314 is mounted in the correct orientation. In some examples, the distal shoulder 310 can match the color of the positioning member 340, e.g., to visually reinforce that the prosthetic valve 314 is in the correct orientation (e.g., matching “white to white”). This configuration advantageously simplifies the process of orienting the prosthetic valve on the delivery device and prevents incorrect orientation, which can result in removal and disposal of the prosthetic valve.

In some examples, as shown, the positioning member 340 can be cylindrical such that fully surrounds the prosthetic valve 314 and is visible to the user in any rotational orientation of the prosthetic valve.

Referring to FIG. 11, as mentioned previously, the inner shaft 304 can comprise a marker band 338 used to align the prosthetic valve 314 (shown as frame 352 in FIG. 11) on the valve mounting portion 322 of the balloon 302. In some examples, this marker band 338 can be a first marker band 338a. The distal end 300 of the delivery apparatus can further comprise two additional marker bands (e.g., radiopaque marker bands): the second marker band 338b and the third marker band 338c.

The second marker band 338b can be disposed on the inner shaft 304 adjacent a proximal end of the balloon 302. In some examples, the marker band 338b can be swaged to an outer diameter of the guidewire lumen inside of the balloon shaft 306. The third marker band 338c can be disposed on a distal end of the outer shaft 328. As shown in FIG. 11, the second marker band 338b can axially align with the proximal end of the balloon 302. During the implantation procedure, fluoroscopy can be used to visualize the second and third marker bands 338b, 338c and determine the position of the outer shaft 328 relative to the balloon 302. This configuration can advantageously ensure that a user retracts the distal end 354 of the outer shaft 328 such that it is no longer over the balloon 302 prior to inflation of the balloon 302. In other words, marker 338b indicates the “pull back distance” for the outer shaft 328. This ensures that the balloon 302 is not constrained during inflation, which could damage the balloon 302 and/or prosthetic valve 314 and/or potentially injure the patient.

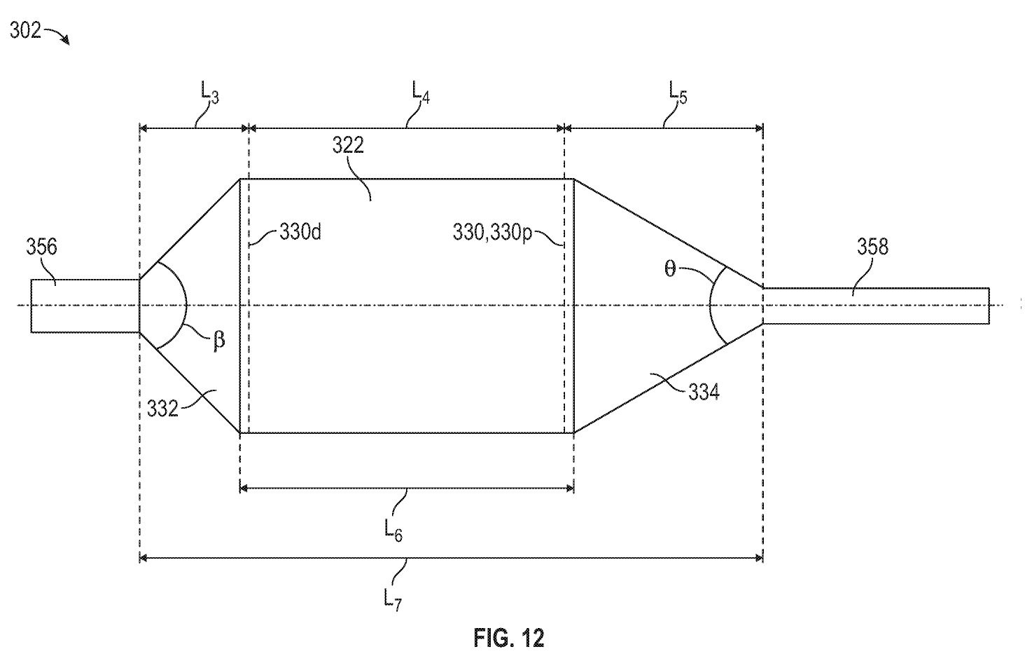

Referring now to FIGS. 12-13, in some examples, the balloon 302 can comprise several portions along the axial length of the balloon 302 that have different lengths. In some examples, portions of the balloon 302 along the axial length of the balloon can also have different thicknesses. FIG. 12 shows the balloon 302 in an inflated state. The balloon 302 comprises a distal end portion 356, a distal cone portion 332, a central portion 322 (also referred to as the “valve mounting portion” or “working length”), a proximal cone portion 334, and a proximal end portion 358. The distal end portion 356 can be coupled to the distal shoulder assembly 310, and the proximal end portion 358 can be coupled to the balloon shaft 306.

The distal cone 332 can have a length L₃, the central portion 322 can have a length L₄, and the proximal cone 334 can have a length L₅. As shown in FIG. 12, in some examples, the length L₃can be shorter than the length L₅. The length L₃ of the distal cone 332 can be minimized to the extent possible in order to minimize the angle of the distal cone without enduring too much stress upon inflation. This advantageously aids in retrieval of the balloon 302 into the outer sheath (e.g., guide sheath 100) post-implantation procedure. In some examples, the distal cone 332 can have a length L₃ of between about 10 mm and about 20 mm, between about 12 mm and about 18 mm, between about 14 mm and about 18 mm, between about 15 mm and about 17 mm. In some examples, the distal cone 332 can have a length L₃between about 15.8 mm and about 17.4 mm.

When inflated, the distal cone 332 can define a distal cone angle β. In some examples, the distal cone angle β can be between about 60 degrees and about 80 degrees, between about 65 degrees and about 75 degrees, between about 70 degrees and about 73 degrees. In some examples, the distal cone angle can be about 71.3 degrees.

The central portion 322 can have a length L₄ configured to be as short as possible while still ensuring that the prosthetic valve 314 can be disposed thereon. The length L₄ can also be referred to as the “working length.” In some examples, the central portion 322 can have a length L₄ between about 30 mm and about 40 mm, between about 31 mm and about 35 mm, between about 31 mm and about 34 mm. In some examples, the central portion 322 can have a length L₄ between about 32.9 mm and about 33.9 mm. The central portion 322 (also referred to as the “valve mounting portion” or “working portion”) can be bounded on the proximal and distal ends by a proximal witness line 330p and a distal witness line 330d, as discussed previously. The buffer distance between the witness lines 330 and the proximal and distal edges, respectively, of the crimped prosthetic heart valve can, for example, help ensure that the valve remains aligned with the central portion 322 during initial inflation (e.g., in some instances, up to or about 15 cc of inflation). After the initial inflation, the prosthetic valve begins to axially foreshorten (and radially expand), which moves the proximal end and/or the distal end of the prosthetic valve away from the proximal witness line 330p and/or the distal witness lines, respectively.

The proximal cone 334 can have a length L₅ that is minimized to the extent possible to generate a selected proximal cone angle θ. The proximal cone angle can be selected to allow adequate balloon expansion and also retrieval into the guide sheath 100 after the inflation process. In some examples, the proximal cone 334 can have a length L₅ between about 12 mm and about 22 mm, between about 15 mm and about 21 mm, between about 16 mm and about 20 mm. In some examples, the proximal cone 334 can have a length L₅ between about 17.6 mm and about 19.2 mm.

When inflated, the proximal cone 334 can define a proximal cone angle θ. In some examples, the proximal cone angle θ can be between about 60 degrees and about 80 degrees, between about 65 degrees and about 75 degrees, between about 66 degrees and about 70 degrees. In some examples, the proximal cone angle θ can be about 68.6 degrees.

The balloon 302 can further have an length L₆ defined as the inflated balloon length of the central portion 322 with an additional safety margin, this is also referred to as the “effective length.” The length L₆ takes into account rounded edges of the balloon formed where the distal cone 332 and the proximal cone 334 meet the central portion 322. When deployed, the prosthetic valve 314 can have a length of approximately 24.5 mm, and when crimped the prosthetic valve can have a length of approximately 31 mm. In some examples, the effective length L₆ can be between about 30 mm and about 45 mm, between about 32 mm and about 40 mm, between about 33 mm and about 37 mm. In some examples, the effective length L₆ can be between about 33.9 mm and about 36.9 mm. This configuration adds a buffer of between about 8 mm and about 12 mm between the effective length L₆ and the length of the deployed prosthetic valve 314. For example, a pre-deployed (i.e., crimped) prosthetic heart valve can have a height of approximately 31 mm, during the initial inflation process. The starting buffer between the witness lines and the proximal and/or distal edges of the crimped prosthetic valve can, for example, help ensure that the prosthetic heart valve remains axially aligned on the balloon during inflation.

Referring to FIG. 13, as mentioned previously, in some examples, the balloon 302 can comprise several portions along the axial length of the balloon that have different thicknesses. For example, the distal and proximal end portions 356, 358 can have a thickness greater than the central portion 322. Thicker balloon walls can advantageously prevent bursting, however, walls that are too thick can cause problems with bonding the balloon to the other components of the delivery apparatus. Accordingly, the dimensions of the inner diameters of the proximal and distal end portions 356, 358 must be selected with these parameters in mind.

The distal end portion 356 can be coupled to the distal shoulder assembly 310, which can require a selected inner diameter ID₁ configured to allow consistent bonding. For example, the inner diameter ID₁ can be between about 0.1 inches to about 0.3 inches, between about 0.15 inches and about 0.25 inches, between about 0.197 inches and about 0.203 inches.

The proximal end portion 358 can be coupled to the balloon shaft 306, which can require a selected inner diameter ID₂ configured to allow consistent bonding. In some examples, as shown in FIG. 13, the inner diameter of the proximal end portion ID₂ can be less than the inner diameter of the distal end portion ID₁. For example, the inner diameter ID₂ can be between about 0.5 inches to about 0.2 inches, from about 0.1 inches to about 0.15 inches, from about 0.118 inches to about 0.126 inches.

Each wall of the central portion 322 of the balloon 302 can have a wall thickness T₁of between about 0.05 inches and about 0.07 inches, about 0.055 inches and about 0.065 inches. In some examples, the wall thickness T₁ can be about 0.063 inches.

In the expanded configuration at 4 atm, the central portion 322 of the balloon 302 can have an outer diameter of between about 25 mm and about 35 mm, between about 28 mm and about 32 mm, between about 29 mm and about 31 mm, between about 29.7 mm and about 30.7 mm. The expanded outer diameter OD of the balloon 302 can be directly tied to the desired dimensions of the expanded prosthetic heart valve 314. For example, the expanded balloon 302 can result in an expanded prosthetic heart valve 314 having an inflow end diameter of about 29.5 mm, an outflow end diameter of about 29.5 mm, and a central diameter of about 27.0 mm.

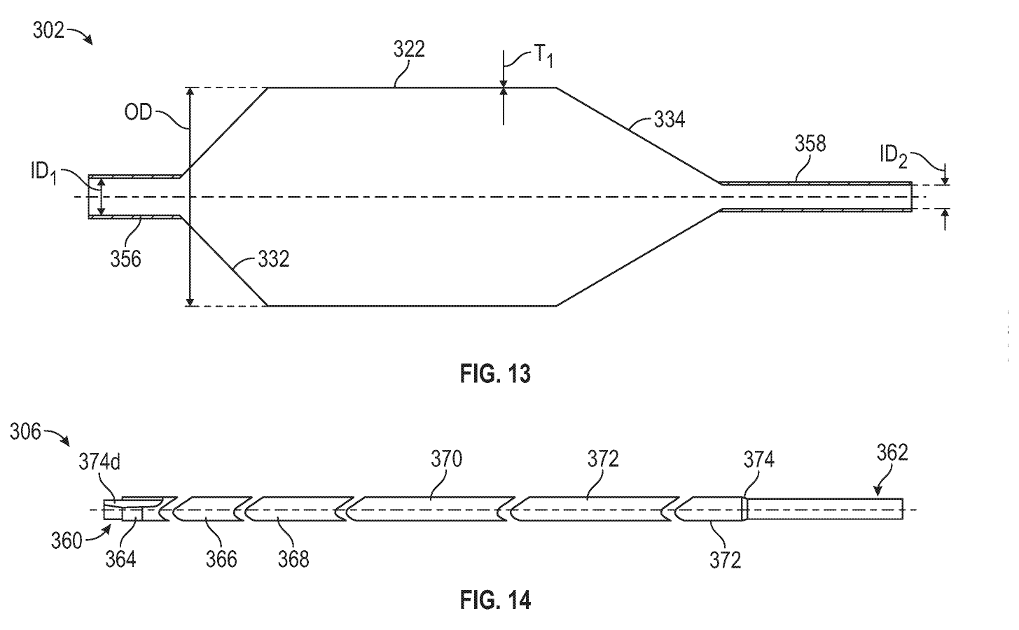

Referring to FIG. 14, as mentioned previously, in some examples, a balloon shaft 306 can be used in lieu of intermediate shaft/balloon shaft 206 in a delivery apparatus, such as delivery apparatus 200. The balloon shaft 306 is configured to allow the prosthetic heart valve 314 to be crimped in an on-balloon configuration. The distal end portion 360 of the shaft 306 is configured with a selected flexibility such that during the prosthetic valve implantation procedure, once the outer shaft 328 is retracted, the balloon shaft 306 is able to maintain the selected position relative to the docking device (if it is an implantation procedure within a docking device such as described with reference to FIGS. 1-4) and/or the native mitral valve. The balloon shaft 306 can also advantageously have a selected rigidity along the axial length of the shaft to ensure that the shaft can be pushed through the native anatomy and also to ensure protection of the inflation lumen within the shaft.

The balloon shaft 306 can comprise a distal end 360 and a proximal end 362. The shaft 306 can comprise a plurality of longitudinally extending sections. For example, the shaft 306 can have a first section 364, a second section 366, a third section 368, a fourth section 370, and a fifth section 372 extending axially along the length of the shaft from the distal end 360 to the proximal end 362. The first section 364, the fourth section 370, and the fifth section 372 can be comparably more rigid than the other sections. The rigidity of the first section 364 can provide support for the distal end portion 360 which is coupled to the balloon 302. The rigidity of the fourth section 370 allows for structural integrity along the length of the section. In some examples, all five sections can comprise thermoplastic elastomers, such as a polyether block amide (PEBA). In some examples, the first and fourth sections 364, 370 can comprise PEBA such as Pebax 6333. In some examples, the fifth section 372 can comprise a blend of polyether block amides (PEBAs) such as Pebax 6333 and Vestamid L21. The rigidity of the fifth section allows for structural integrity along the length of the section.

The second section 366 can be relatively more flexible than all other sections, which advantageously allows for ease of tracking through the patient’s native vasculature and positioning the distal end portion 360 of the shaft within the docking device/native annulus. In some examples, the second section comprises PEBA, such as Pebax 3533.

The third section 368 can be semi-rigid relative to the other portions. For example, the third section 368 can be more rigid than the second section 366, but less rigid than the first, fourth, and fifth sections 364, 370, and 372. The semi-rigidity of the third section 368 allows for support of the shaft 306 after the outer shaft 328 has been retracted. In some examples, the third section 368 comprises PEBA, such as Pebax 5533.

In some examples, the first section 364 can have a length between about 0.05 inches and about 0.3 inches long, between about 0.1 inches and about 0.15 inches, between about 0.12 inches and about 0.14 inches. In some examples, the length of the first section 364 can be about 0.13 inches. In some examples, the second section 366 can have a length between about 2.0 inches and about 3.0 inches, between about 2.2 inches and about 2.8 inches, between about 2.4 inches and about 2.6 inches. In some examples, the length of the second section 366 can be about 2.5 inches. In some examples, the third section 368 can have a length between about 2.0 inches and about 3.0 inches, between about 2.2 inches and about 2.8 inches, between about 2.4 inches and about 2.6 inches. In some examples, the length of the third section 368 can be about 2.5 inches. In some examples, the fourth section 370 can have a length between about 4.0 inches and about 6.0 inches, between about 4.5 inches and about 5.5 inches, between about 4.9 inches and about 5.1 inches. In some examples, the length of the fourth section 370 can be 5.0 inches. In some examples, the fifth section 372 can have a length between about 44.0 inches and about 46.0 inches, between about 44.5 inches and about 45.5 inches, between about 44.9 inches and about 45.1 inches. In some examples, the length of the fifth section 372 can be 45.0 inches.

As shown in FIG. 14, the fifth section 372 can comprise one or more transition sections 374. The transition sections 374 can taper from a first diameter at a distal end thereof to a second, smaller diameter at a proximal end thereof. The transition sections 374 can be configured to gradually decrease the outer diameter of the fifth section 372 to allow for bonding with a adaptor (such as adaptor 212 described previously) at a proximal end portion 362 of the balloon shaft 306. The first section 364 can also comprise a transition section 374d (referred to as the “distal transition section”) having a distal diameter smaller than a diameter of the first section 364 to facilitate bonding with the proximal end portion of the balloon 302. In some examples, the distal transition section 374d can have a length between about 0.9 inches and about 0.15 inches, between about 0.1 inches and about 0.14 inches, between about 0.11 inches and about 0.13 inches. In some examples, the length of the distal transition section 374d can be 0.12 inches.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus, comprising: a handle; a first shaft extending distally from the handle; a second shaft extending distally from the handle coaxially through the first shaft; a shoulder assembly disposed at a distal end portion of the delivery apparatus; and a balloon coupled to a distal end of the second shaft and extending over the shoulder assembly, the balloon being shape set to form a distal balloon bump over the shoulder assembly, a central portion, and a proximal balloon bump disposed proximally of the central portion.

Example 2. The delivery apparatus of any example herein, particularly example 1, further comprising a third shaft extending distally from the handle coaxially through the first and second shafts.

Example 3. The delivery apparatus of any example herein, particularly any one of examples 1-2, wherein the shoulder assembly is coupled to a distal end portion of the third shaft.

Example 4. The delivery apparatus of any example herein, particularly any one of examples 1-3, further comprising a radially expandable and compressible prosthetic valve disposed at least partially over the central portion of the balloon in a radially compressed configuration.

Example 5. The delivery apparatus of any example herein, particularly example 4, wherein an inflow end portion of the prosthetic valve is crimped partially over a distal end portion of the proximal balloon bump.

Example 6. The delivery apparatus of any example herein, particularly any one of examples 4-5, wherein the distal balloon bump has a width greater than a width of the radially compressed prosthetic valve.

Example 7. The delivery apparatus of any example herein, particularly any one of examples 4-6, wherein the prosthetic valve comprises a positioning member disposed on an outflow end of the prosthetic valve, and wherein when the prosthetic valve is disposed on the balloon the positioning member is adjacent the shoulder assembly.

Example 8. The delivery apparatus of any example herein, particularly any one of examples 1-7, wherein the proximal balloon bump has a width of 7.0 mm.

Example 9. The delivery apparatus of any example herein, particularly any one of examples 1-8, wherein the distal balloon bump has a width of 8.75 mm.

Example 10. The delivery apparatus of any example herein, particularly any one of examples 1-9, wherein the central portion has a width of 4.3 mm.

Example 11. The delivery apparatus of any example herein, particularly any one of examples 1-10, wherein the balloon comprises a distal witness line disposed at a distal end of the central portion and a proximal witness line disposed at a proximal end of the central portion.

Example 12. The delivery apparatus of any example herein, particularly example 11, wherein the distal witness line is spaced 1.25 mm from a proximal edge of the shoulder assembly.

Example 13. The delivery apparatus of any example herein, particularly any one of examples 1-12, wherein the balloon comprises a distal cone portion and a proximal cone portion.

Example 14. The delivery apparatus of any example herein, particularly example 13, wherein the distal cone has a first length shorter than a second length of the proximal cone.

Example 15. The delivery apparatus of any example herein, particularly any one of examples 13-14, wherein the first length is between 15.8 mm and 17.4 mm.

Example 16. The delivery apparatus of any example herein, particularly any one of examples 13-15, wherein the second length is between 17.6 mm and 19.2 mm.

Example 17. The delivery apparatus of any example herein, particularly any one of examples 13-16, wherein the balloon defines an effective length including a central portion disposed between the distal cone portion and the proximal cone portion, and wherein the effective length is between 30 mm and 45 mm.

Example 18. The delivery apparatus of any example herein, particularly any one of examples 13-17, wherein the balloon further comprises a distal end portion disposed distal to the distal cone portion, a proximal end portion disposed proximal to the proximal cone portion, and the central portion disposed between the distal cone portion and the proximal cone portion, and wherein the proximal and distal end portions have a wall thickness that is greater than a wall thickness of the central portion.

Example 19. The delivery apparatus of any example herein, particularly example 18, wherein an inner diameter of the distal end portion is between 0.1 inches and 0.3 inches.

Example 20. The delivery apparatus of any example herein, particularly any one of examples 18-19, wherein an inner diameter of the proximal end portion is between 0.5 inches and 0.2 inches.

Example 21. The delivery apparatus of any example herein, particularly any one of examples 18-20, wherein the central portion has a wall thickness of between 0.05 inches and 0.07 inches.

Example 22. The delivery apparatus of any example herein, particularly any one of examples 18-21, wherein the central portion has a wall thickness of 0.063 inches.

Example 23. The delivery apparatus of any example herein, particularly any one of examples 18-22, wherein the central portion has an outer diameter of between 25 mm and 35 mm.

Example 24. The delivery apparatus of any example herein, particularly any one of examples 1-23, wherein the second shaft comprises a plurality of longitudinally extending sections including a first section, a second section, a third section, a fourth section, and a fifth section.

Example 25. The delivery apparatus of any example herein, particularly example 24, wherein the first section is more rigid than the second, third, fourth, and fifth sections.

Example 26. The delivery apparatus of any example herein, particularly any one of examples 24-25, wherein the first, fourth, and fifth sections are more rigid than the second and third sections.

Example 27. The delivery apparatus of any example herein, particularly any one of examples 24-26, wherein the first and fourth sections comprise Pebax 6333.

Example 28. The delivery apparatus of any example herein, particularly any one of examples 24-27, wherein the fifth section comprises Pebax 6333 and Vestamid L21.

Example 29. The delivery apparatus of any example herein, particularly any one of examples 24-28, wherein the second section comprises Pebax 3533.

Example 30. The delivery apparatus of any example herein, particularly any one of examples 24-29, wherein the third section comprises Pebax 5533.

Example 31. The delivery apparatus of any example herein, particularly any one of examples 24-30, wherein the first section has a length between 0.05 inches and 0.3 inches.

Example 32. The delivery apparatus of any example herein, particularly any one of examples 24-31, wherein the first section has a length of 0.13 inches.

Example 33. The delivery apparatus of any example herein, particularly any one of examples 24-32, wherein the second section has a length between 2.0 inches and 3.0 inches.

Example 34. The delivery apparatus of any example herein, particularly any one of examples 24-33, wherein the second section has a length of 2.5 inches.

Example 35. The delivery apparatus of any example herein, particularly any one of examples 24-34, wherein the third section has a length between 2.0 inches and 3.0 inches.

Example 36. The delivery apparatus of any example herein, particularly any one of examples 24-35, wherein the third section has a length of 2.5 inches.

Example 37. The delivery apparatus of any example herein, particularly any one of examples 24-36, wherein the fourth section has a length between 4.0 inches and 6.0 inches.

Example 38. The delivery apparatus of any example herein, particularly any one of examples 24-37, wherein the fourth section has a length of 5.0 inches.

Example 39. The delivery apparatus of any example herein, particularly any one of examples 24-38, wherein the fifth section has a length between 44.0 inches and 46.0 inches.

Example 40. The delivery apparatus of any example herein, particularly any one of examples 24-39, wherein the fifth section has a length of 45.0 inches.

Example 41. The delivery apparatus of any example herein, particularly any one of examples 24-40, wherein the fifth section comprises one or more transition sections tapering from a first diameter at a distal end thereof to a second, smaller diameter at a proximal end thereof.

Example 42. The delivery apparatus of any example herein, particularly any one of examples 1-41, the second shaft further comprising a proximal end portion that extends proximally from the handle and is coupled to an adaptor.

Example 43. The delivery apparatus of any example herein, particularly example 42, wherein the adaptor comprises a port configured to receive fluid therethrough and fluidly coupled to an inner lumen of the second shaft.

Example 44. A delivery apparatus, comprising: a handle; a first shaft extending distally from the handle; a second shaft extending distally from the handle coaxially through the first shaft; a third shaft extending distally from the handle coaxially through the first and second shafts; a shoulder assembly coupled to a distal end portion of the third shaft; and a balloon coupled to a distal end of the second shaft and extending over the shoulder assembly, the balloon being shape set to form a distal balloon bump over the shoulder assembly, a central portion, and a proximal balloon bump disposed proximally of the central portion.

Example 45. The delivery apparatus of any example herein, particularly example 44, wherein the first shaft comprises a first marker band disposed at a distal end portion thereof, and the second shaft comprises a second marker band disposed at a distal end portion thereof adjacent a proximal end of the balloon.

Example 46. The delivery apparatus of any example herein, particularly example 45, further comprising a third marker band disposed on the third shaft adjacent the shoulder assembly, the third marker band being positioned a selected distance away from a proximal end of the shoulder assembly.

Example 47. The delivery apparatus of any example herein, particularly any one of examples 44-46, further comprising a radially expandable and compressible prosthetic valve disposed at least partially over the central portion of the balloon in a radially compressed configuration.

Example 48. The delivery apparatus of any example herein, particularly example 47, wherein an inflow end portion of the prosthetic valve is crimped partially over a distal end portion of the proximal balloon bump.

Example 49. The delivery apparatus of any example herein, particularly any one of examples 47-48, wherein the distal balloon bump has a width greater than a width of the radially compressed prosthetic valve.

Example 50. The delivery apparatus of any example herein, particularly any one of examples 47-49, wherein the prosthetic valve comprises a positioning member disposed on an outflow end of the prosthetic valve, and wherein when the prosthetic valve is disposed on the balloon the positioning member is adjacent the shoulder assembly.

Example 51. The delivery apparatus of any example herein, particularly any one of examples 44-50, wherein the proximal balloon bump has a width of 7.0 mm.

Example 52. The delivery apparatus of any example herein, particularly any one of examples 44-51, wherein the distal balloon bump has a width of 8.75 mm.

Example 53. The delivery apparatus of any example herein, particularly any one of examples 44-52, wherein the central portion has a width of 4.3 mm.

Example 54. The delivery apparatus of any example herein, particularly any one of examples 44-53, wherein the balloon comprises a distal witness line disposed at a distal end of the central portion and a proximal witness line disposed at a proximal end of the central portion.

Example 55. The delivery apparatus of any example herein, particularly example 54, wherein the distal witness line is spaced 1.25 mm from a proximal edge of the shoulder assembly.

Example 56. The delivery apparatus of any example herein, particularly any one of examples 44-55, wherein the balloon comprises a distal cone portion and a proximal cone portion.

Example 57. The delivery apparatus of any example herein, particularly example 56, wherein the distal cone has a first length shorter than a second length of the proximal cone.

Example 58. The delivery apparatus of any example herein, particularly any one of examples 56-57, wherein the first length is between 15.8 mm and 17.4 mm.

Example 59. The delivery apparatus of any example herein, particularly any one of examples 56-58, wherein the second length is between 17.6 mm and 19.2 mm.

Example 60. The delivery apparatus of any example herein, particularly any one of examples 56-59, wherein the balloon defines an effective length including a central portion disposed between the distal cone portion and the proximal cone portion, and wherein the effective length is between 30 mm and 45 mm.

Example 61. The delivery apparatus of any example herein, particularly any one of examples 56-60, wherein the balloon further comprises a distal end portion disposed distal to the distal cone portion, a proximal end portion disposed proximal to the proximal cone portion, and the central portion disposed between the distal cone portion and the proximal cone portion, and wherein the proximal and distal end portions have a wall thickness that is greater than a wall thickness of the central portion.

Example 62. The delivery apparatus of any example herein, particularly example 61, wherein an inner diameter of the distal end portion is between 0.1 inches and 0.3 inches.

Example 63. The delivery apparatus of any example herein, particularly any one of examples 61-62, wherein an inner diameter of the proximal end portion is between 0.5 inches and 0.2 inches.

Example 64. The delivery apparatus of any example herein, particularly any one of examples 61-63, wherein the central portion has a wall thickness of between 0.05 inches and 0.07 inches.

Example 65. The delivery apparatus of any example herein, particularly any one of examples 61-64, wherein the central portion has a wall thickness of 0.063 inches.

Example 66. The delivery apparatus of any example herein, particularly any one of examples 61-65, wherein the central portion has an outer diameter of between 25 mm and 35 mm.

Example 67. The delivery apparatus of any example herein, particularly any one of examples 44-66, wherein the second shaft comprises a plurality of longitudinally extending sections including a first section, a second section, a third section, a fourth section, and a fifth section.

Example 68. The delivery apparatus of any example herein, particularly example 67, wherein the first section is more rigid than the second, third, fourth, and fifth sections.

Example 69. The delivery apparatus of any example herein, particularly any one of examples 67-68, wherein the first, fourth, and fifth sections are more rigid than the second and third sections.

Example 70. The delivery apparatus of any example herein, particularly any one of examples 67-69, wherein the first and fourth sections comprise Pebax 6333.

Example 71. The delivery apparatus of any example herein, particularly any one of examples 67-70, wherein the fifth section comprises Pebax 6333 and Vestamid L21.

Example 72. The delivery apparatus of any example herein, particularly any one of examples 67-71, wherein the second section comprises Pebax 3533.

Example 73. The delivery apparatus of any example herein, particularly any one of examples 67-72, wherein the third section comprises Pebax 5533.

Example 74. The delivery apparatus of any example herein, particularly any one of examples 67-73, wherein the first section has a length between 0.05 inches and 0.3 inches.

Example 75. The delivery apparatus of any example herein, particularly any one of examples 67-74, wherein the first section has a length of 0.13 inches.

Example 76. The delivery apparatus of any example herein, particularly any one of examples 67-75, wherein the second section has a length between 2.0 inches and 3.0 inches.

Example 77. The delivery apparatus of any example herein, particularly any one of examples 67-76, wherein the second section has a length of 2.5 inches.

Example 78. The delivery apparatus of any example herein, particularly any one of examples 67-77, wherein the third section has a length between 2.0 inches and 3.0 inches.

Example 79. The delivery apparatus of any example herein, particularly any one of examples 67-78, wherein the third section has a length of 2.5 inches.

Example 80. The delivery apparatus of any one of claims 67-79, wherein the fourth section has a length between 4.0 inches and 6.0 inches.

Example 81. The delivery apparatus of any example herein, particularly any one of examples 67-80, wherein the fourth section has a length of 5.0 inches.

Example 82. The delivery apparatus of any example herein, particularly any one of examples 67-81, wherein the fifth section has a length between 44.0 inches and 46.0 inches.

Example 83. The delivery apparatus of any example herein, particularly any one of examples 67-82, wherein the fifth section has a length of 45.0 inches.

Example 84. The delivery apparatus of any example herein, particularly any one of examples 67-83, wherein the fifth section comprises one or more transition sections tapering from a first diameter at a distal end thereof to a second, smaller diameter at a proximal end thereof.

Example 85. The delivery apparatus of any example herein, particularly any one of examples 44-84, the second shaft further comprising a proximal end portion that extends proximally from the handle and is coupled to an adaptor.

Example 86. The delivery apparatus of any example herein, particularly example 85, wherein the adaptor comprises a port configured to receive fluid therethrough and fluidly coupled to an inner lumen of the second shaft.

Example 87. A delivery assembly, comprising: a guide catheter comprising a handle and a guide shaft extending distally from the handle; and a delivery apparatus configured to be advanced through the guide catheter, the delivery apparatus comprising: a handle; a first shaft extending distally from the handle; a second shaft extending distally from the handle coaxially through the first shaft; a third shaft extending distally from the handle coaxially through the first and second shafts; a shoulder assembly coupled to a distal end portion of the third shaft; and a balloon coupled to a distal end of the second shaft and extending over the shoulder assembly, the balloon being shape set to form a distal balloon bump over the shoulder assembly, a central portion, and a proximal balloon bump disposed proximally of the central portion.

Example 88. A delivery assembly, comprising: a delivery apparatus comprising: a handle; a first shaft extending distally from the handle; a second shaft extending distally from the handle coaxially through the first shaft; a third shaft extending distally from the handle coaxially through the first and second shafts; a shoulder assembly coupled to a distal end portion of the third shaft; a balloon coupled to a distal end of the second shaft and extending over the shoulder assembly, the balloon being shape set to form a distal balloon bump over the shoulder assembly, a central portion, and a proximal balloon bump disposed proximally of the central portion; and a radially expandable and compressible prosthetic heart valve, the heart valve comprising a frame and a valvular structure disposed within and supported by the frame; wherein the prosthetic heart valve is crimped over the central portion of the balloon in a radially compressed configuration such that an inflow end portion of the prosthetic heart valve is crimped partially over the proximal balloon bump.

Example 89. The delivery apparatus of any example herein, particularly example 88, wherein the distal balloon bump has a width greater than a width of the radially compressed prosthetic heart valve crimped on the central portion of the balloon.

Example 90. The delivery apparatus of any example herein, particularly any one of examples 88-89, wherein the prosthetic heart valve comprises a positioning member disposed on an outflow end of the prosthetic valve, wherein when the prosthetic heart valve is disposed on the balloon the positioning member is adjacent the shoulder assembly.

Example 91. A delivery apparatus, comprising: a handle; a first shaft extending distally from the handle; and a second shaft extending distally from the handle coaxially through the first shaft, the second shaft comprising a plurality of longitudinally extending sections including a first section, a second section, a third section, a fourth section, and a fifth section; wherein the first section is more rigid than the second, third, fourth, and fifth sections; and wherein the first, fourth, and fifth sections are more rigid than the second and third sections.

Example 92. The delivery apparatus of any example herein, particularly example 91, wherein the first and fourth sections comprise Pebax 6333.

Example 93. The delivery apparatus of any example herein, particularly any one of examples 91-92, wherein the fifth section comprises Pebax 6333 and Vestamid L21.

Example 94. The delivery apparatus of any example herein, particularly any one of examples 91-93, wherein the second section comprises Pebax 3533.

Example 95. The delivery apparatus of any example herein, particularly any one of examples 91-94, wherein the third section comprises Pebax 5533.

Example 96. The delivery apparatus of any example herein, particularly any one of examples 91-95, wherein the first section has a length between 0.05 inches and 0.3 inches.

Example 97. The delivery apparatus of any example herein, particularly any one of examples 91-96, wherein the first section has a length of 0.13 inches.

Example 98. The delivery apparatus of any example herein, particularly any one of examples 91-97, wherein the second section has a length between 2.0 inches and 3.0 inches.

Example 99. The delivery apparatus of any example herein, particularly any one of examples 91-98, wherein the second section has a length of 2.5 inches.

Example 100. The delivery apparatus of any example herein, particularly any one of examples 91-99, wherein the third section has a length between 2.0 inches and 3.0 inches.

Example 101. The delivery apparatus of any example herein, particularly any one of examples 91-100, wherein the third section has a length of 2.5 inches.

Example 102. The delivery apparatus of any example herein, particularly any one of examples 91-101, wherein the fourth section has a length between 4.0 inches and 6.0 inches.

Example 103. The delivery apparatus of any example herein, particularly any one of examples 91-102, wherein the fourth section has a length of 5.0 inches.

Example 104. The delivery apparatus of any example herein, particularly any one of examples 91-103, wherein the fifth section has a length between 44.0 inches and 46.0 inches.

Example 105. The delivery apparatus of any example herein, particularly any one of examples 91-104, wherein the fifth section has a length of 45.0 inches.

Example 106. The delivery apparatus of any example herein, particularly any one of examples 91-105, wherein the fifth section comprises one or more transition sections tapering from a first diameter at a distal end thereof to a second, smaller diameter at a proximal end thereof.

Example 107. The delivery apparatus of any example herein, particularly any one of examples 91-106, the second shaft further comprising a proximal end portion that extends proximally from the handle and is coupled to an adaptor.

Example 108. The delivery apparatus of any example herein, particularly, any one of examples 4-7, 47-50, and 88-90 wherein the prosthetic heart valve is sterilized.

Example 109. The delivery apparatus of any example herein, particularly, any one of examples 1-107, wherein the delivery apparatus is sterilized.

The features described herein with regard to any example can be combined with other features described in any one or more of the other examples, unless otherwise stated. For example, any one or more of the features of a delivery apparatus can be combined with any one or more other features, or with a prosthetic heart valve having any of the features described herein. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A delivery apparatus, comprising:
a handle;
a first shaft extending distally from the handle;
a second shaft extending distally from the handle coaxially through the first shaft;
a shoulder assembly disposed at a distal end portion of the delivery apparatus; and
a balloon coupled to a distal end of the second shaft and extending over the shoulder assembly, the balloon being shape set to form a distal balloon bump over the shoulder assembly, a central portion, and a proximal balloon bump disposed proximally of the central portion.

2. The delivery apparatus of claim 1, further comprising a third shaft extending distally from the handle coaxially through the first and second shafts, and wherein the shoulder assembly is coupled to a distal end portion of the third shaft.

3. The delivery apparatus of claim 1, further comprising a radially expandable and compressible prosthetic valve disposed at least partially over the central portion of the balloon in a radially compressed configuration, and wherein an inflow end portion of the prosthetic valve is crimped partially over a distal end portion of the proximal balloon bump.

4. The delivery apparatus of claim 3, wherein the distal balloon bump has a width greater than a width of the radially compressed prosthetic valve.

5. The delivery apparatus of claim 1, wherein the balloon comprises a distal witness line disposed at a distal end of the central portion and a proximal witness line disposed at a proximal end of the central portion.

6. The delivery apparatus of claim 1, wherein the balloon comprises a distal cone portion and a proximal cone portion.

7. The delivery apparatus of claim 6, wherein the distal cone has a first length shorter than a second length of the proximal cone.

8. The delivery apparatus of claim 7, wherein the balloon defines an effective length including a central portion disposed between the distal cone portion and the proximal cone portion, and wherein the effective length is between 30 mm and 45 mm.

9. The delivery apparatus of claim 7, wherein the balloon further comprises a distal end portion disposed distal to the distal cone portion, a proximal end portion disposed proximal to the proximal cone portion, and the central portion disposed between the distal cone portion and the proximal cone portion, and wherein the proximal and distal end portions have a wall thickness that is greater than a wall thickness of the central portion.

10. The delivery apparatus of claim 9, wherein an inner diameter of the distal end portion is between 0.1 inches and 0.3 inches.

11. The delivery apparatus of claim 9, wherein an inner diameter of the proximal end portion is between 0.5 inches and 0.2 inches.

12. The delivery apparatus of claim 9, wherein the central portion has a wall thickness of between 0.05 inches and 0.07 inches.

13. The delivery apparatus of claim 9, wherein the central portion has an outer diameter of between 25 mm and 35 mm.

14. The delivery apparatus of claim 1, wherein the second shaft comprises a plurality of longitudinally extending sections including a first section, a second section, a third section, a fourth section, and a fifth section, and wherein the first section is more rigid than the second, third, fourth, and fifth sections.

15. The delivery apparatus of claim 14, wherein the first, fourth, and fifth sections are more rigid than the second and third sections.

16. The delivery apparatus of claim 14, wherein the fifth section comprises one or more transition sections tapering from a first diameter at a distal end thereof to a second, smaller diameter at a proximal end thereof.

17. The delivery apparatus of claim 1, the second shaft further comprising a proximal end portion that extends proximally from the handle and is coupled to an adaptor, and wherein the adaptor comprises a port configured to receive fluid therethrough and fluidly coupled to an inner lumen of the second shaft.
